(19) United States
(12) Patent Application Publication
(10) Pub. No.: US 2025/0152711 A1
(43) Pub. Date: May 15, 2025

(54) COMPOSITIONS AND METHODS FOR MODULATING THE IMMUNE SYSTEM

(71) Applicants: RION INC., Rochester, MN (US); MAYO FOUNDATION FOR MEDICAL EDUCATION AND RESEARCH, Rochester, MN (US)

(72) Inventors: Atta Behfar, Rochester, MN (US); Timothy Peterson, Rochester, MN (US); Laura Becher, Rochester, MN (US); Brooke Paradise, Rochester, MN (US); Christopher Paradise, Mantorville, MN (US)

(21) Appl. No.: 18/838,945

(22) PCT Filed: Feb. 24, 2023
(86) PCT No.: PCT/US2023/013820

§ 371 Date: Aug. 15, 2024

Related U.S. Application Data
(60) Provisional application No. 63/313,579, filed on Feb. 24, 2022.

(51) Int. Cl. A61K 40/17; C12N 5/0786

(52) U.S. Cl. CPC A61K 40/17; C12N 5/0645; C12N 2502/1171

(57) ABSTRACT

Compositions and methods of modulating the immune system generally involve immunomodulatory exosomes derived from platelet rich plasma subjected to multiple freeze-thaw cycles and lyophilization. In one or more embodiments, the methods involve administering the composition to a subject in an amount effective to modulate the subject's immune system. In other embodiments, the methods involve culturing a hematopoietic progenitor cell or cell differentiated from a hematopoietic progenitor cell with an immunomodulatory exosome to produce treated cells. The treated cells are then administered to the subject in an amount effective to modulate the subject's immune system.

### CROSS-REFERENCE TO RELATED APPLICATIONS

This application claims the benefit of U.S. Provisional Application Ser. No. 63/313,579, filed on Feb. 24, 2022, which is incorporated by reference herein in its entirety.

### SUMMARY

This disclosure describes compositions and methods that modulate the immune system of a subject.

In one aspect, this disclosure describes a method that generally includes culturing a hematopoietic progenitor cell or a cell differentiated from a hematopoietic progenitor cell with an immunomodulatory exosome to produce a treated cell. The immunomodulatory exosome (e.g., purified exosome product, “PEP”) is derived from platelet rich plasma subjected to multiple freeze-thaw cycles and lyophilization.

In one aspect, this disclosure describes that a hematopoietic progenitor cell is a myeloid progenitor cell. In one or more embodiments, the hematopoietic progenitor cell is a lymphocyte progenitor cell. In one or more other embodiments, the hematopoietic progenitor cell is a peripheral blood mononuclear cell (PBMC).

In one or more embodiments, the treated cell can be a monocyte or a macrophage.

In another aspect, this disclosure describes therapeutic methods in which a treated cell is administered to a subject. In one or more embodiments, the subject is suffering from an inflammation-associated disease state.

In one or more embodiments, the treated cell administered to the subject is an M2 macrophage. In one or more of these embodiments, the M2 macrophage expresses CD163.

In one or more embodiments, the treated cell expresses CD14 or CD206 or both.

In one or more embodiments, the treated cell expresses IL-10, TGF-β, IL-1ra, or Arginase 1, or a combination thereof.

In another aspect, this disclosure describes a composition for use in treating an inflammation-associated disease state in a subject. Generally, the composition includes immunomodulatory exosomes derived from platelet rich plasma subjected to multiple freeze-thaw cycles and lyophilization.

In one or more embodiments, the composition is formulated for intravenous administration. In one or more embodiments, the composition is formulated for intramuscular administration. In one or more embodiments, the composition is formulated for intraperitoneal administration.

In another aspect, this disclosure describes a method of treating an inflammation-associated disease state in a subject. Generally, the method includes administering to the subject a composition that includes immunomodulatory exosomes derived from platelet rich plasma subjected to multiple freeze-thaw cycles and lyophilization.

In one or more embodiments, the composition is administered intravenously. In one or more embodiments, the composition is administered intramuscularly. In one or more embodiments, the composition is administered intraperitoneally.

In one or more embodiments, administering the composition to the subject promotes expansion of Treg cells. In some of these embodiments, the Treg cells have a phenotype comprising CD3⁺CD4⁺CD25^{hi}FoxP3⁺. In some of these embodiments, the Treg cells have a phenotype that further includes CD127^{lo}.

In one or more embodiments, administering the composition to the subject promotes expansion of M2 macrophages. In some of these embodiments, the M2 macrophages express CD163. In some of these embodiments, the M2 macrophages do not express a detectable level of CD80. In one or more embodiments, the M2 macrophages express CD14, CD206, or both.

In one or more embodiments, the method further includes co-administering an additional therapeutic agent. In one or more of these embodiments, the additional therapeutic agent includes an immunomodulatory monoclonal antibody or immunomodulatory small-molecule.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Overview of purified exosome product (PEP) use cases in the context of immunomodulation. In vivo or in vitro exposure to PEP modulates the differentiation capacity and/or phenotype of cell types derived from hematopoietic progenitor cells including but not limited to monocytes, macrophages (MΦ), microglia, and T cells.

FIG. 2. Scanning electron microscopy (SEM) images of PEP binding to immune cells. (A) T cell; (B) T cell (high magnification); (C) monocyte.

FIG. 3. Schematic of in vitro culture system (DEVERRA THERAPEUTICS, Seattle, Washington). (A) Immobilized engineered Delta Like Canonical Notch Ligand 1 (Delta1^{ext-IgG}) is used for expanding and differentiating hematopoietic stem/progenitor cells (HSPCs). (B) Schematic depicting the immobilized Delta1^{ext-IgG} construct interacting with Notch receptor on hematopoietic stem/progenitor cell to promote expansion of differentiated cell types.

FIG. 4. Murine-derived monocytes were cultured in vitro with media only (control), media plus PEP (PEP), media plus lipopolysaccharide (LPS), and media plus lipopolysaccharide and PEP (LPS+PEP). PEP reduces the expression of pro-inflammatory cytokines typically secreted by M1-macrophages in response to LPS exposure.

FIG. 5. Murine-derived monocytes were cultured in vitro with media only (control), media plus PEP (PEP), media plus lipopolysaccharide (LPS), and media plus lipopolysaccharide and PEP (LPS+PEP). PEP increases the expression of markers associated with M2-macrophages.

FIG. 6. Murine-derived monocytes were cultured in vitro with media only (control) or media plus PEP (PEP). Monocyte proliferation was stimulated with PEP, as measured by Ki-67 expression.

FIG. 7. Murine-derived macrophages were cultured with media only (control), media plus PEP (PEP), media plus lipopolysaccharide (LPS), and media plus lipopolysaccharide and PEP (LPS+PEP) and assessed via flow cytometry. LPS induces the pro-inflammatory M1-associated cytokine, IL-1β. The expression of IL-1β due to LPS is reduced in the presence of PEP (top). LPS alone reduces the expression of the M2-associated marker, CD206. In the presence of PEP, CD206 expression increases in LPS-treated macrophages (bottom).

FIG. 8. Confocal microscopy analysis of murine-derived macrophages of FIG. 7. (A) IL-1β expression in LPS-stimulated macrophages. (B) Co-culture of LPS-treated macrophages with PEP increases expression of CD206.

FIG. 9. An acute myocardial infarction (MI) was induced in a porcine model. MI was treated with stent placement only (Infarction) or stent placement plus PEP administration (MI+PEP). mRNA expression analysis was conducted on tissues derived from porcine cardiac tissue in both groups.

FIG. 10. Assessment of the ratio of M2 macrophage phenotype (CD163) to mature macrophage population (25F9) following induced acute myocardial infarction. (A) immunohistochemistry and confocal microscopy; (B) quantified via ImageJ analysis.

FIG. 11. Immunohistochemistry of porcine urethral tissue for general macrophages (Mϕ), CD163 M2 specific macrophages, and DAPI. Merge overlay highlights PEP samples contained co-localization of macrophages and CD163⁺ staining of tissues. (n=12, 15, 20) Scale=20 μm.

FIG. 12. ImageJ blinded quantification of immunohistochemistry staining in FIG. 11. M2 specific macrophage: M1 specific macrophage (M1:M2) ratio was determined from the comparison of image area of M2:(Mϕ-M2). A Haldane correction was utilized to account for division of 0 in M2:M1 ratio. The correction adds 0.5 to all values in the list to allow for the calculation to not yield an error. The graph represents the raw ratio of M2:M1 macrophages. Red=statistical outlier. Dotted line at y=1 shows the value at which the ratio would be equal. The log base 2 scale shows a M2:M1>1 at values Log2>0 and M2:M1<1 at values Log2<0. ***=p<0.05. Red=statistical outlier. Dotted line at y=0 shows the value at which the ratio would be equal. (n=12, 15, 20)

FIG. 13. Chemotaxis of polarized microglia pretreated with LPS, IL-4, or untreated displayed no migration into the lower reservoirs without PEP.

FIG. 14. Chemotaxis of polarized microglia pretreated with LPS for 24 hours resulting in an M1 phenotype, displayed negligible chemotaxis towards the bottom reservoir of PEP-supplemented media.

FIG. 15. Chemotaxis of polarized microglia pretreated with IL-4 to induce an M2 phenotype displayed dose-dependent chemotaxis towards PEP.

FIG. 16. Chemotaxis of untreated naïve microglia (M0) displayed strong dose-dependent chemotaxis toward PEP.

FIG. 17. Proliferative effects of PEP on polarized microglia. Microglia were polarized into three categories: M1 (LPS-pretreated), M2 (IL-4-pretreated), and M0 (untreated). After polarization, microglia were treated with 10% PEP in serum-free media. M2 and M0 displayed increased proliferation with PEP treatment compared to M1 microglia.

FIG. 18. Flow cytometry results of T cell proliferation assay. (A) In the absence of T cell stimulation, PEP did not impact the number of T cells. (B) Upon stimulation of T cells with CD3/CD28, CD4 T cell proliferation was inhibited by the presence of PEP.

### DETAILED DESCRIPTION OF ILLUSTRATIVE EMBODIMENTS

This disclosure describes compositions and methods to modulate the immune system of a subject. The methods generally exploit immunomodulatory properties of immunomodulatory exosomes derived from platelet rich plasma and subjected to multiple freeze-thaw cycles and lyophilization. In one or more methods, a composition that includes the immunomodulatory exosome may be administered directly to a subject in an amount effective to modulate the immune system of the subject. In one or more other methods, hematopoietic progenitor cells or cells differentiated from hematopoietic progenitor cells are cultured with immunomodulatory exosomes to produce treated cells. The treated cells may be administered to the subject in an amount effective to modulate the immune system of the subject.

In brief, this disclosure demonstrates the ability of immunomodulatory exosomes to bind and modulate immune cells. Particularly, immunomodulatory exosomes support the polarization of macrophages toward an M2 anti-inflammatory phenotype and the reduction of an M1 pro-inflammatory profile both in vitro and in vivo. This is evidenced by alterations in cytokine production and gene expression. Additionally, the immunomodulatory exosomes (e.g., PEP) recruit M0 and M2 polarized microglia and supports their proliferation. Furthermore, the immunomodulatory exosomes inhibit the proliferation of activated CD4+ T cells. Therefore, immunomodulatory exosomes may be used to support the reduction of inflammation in clinical disease.

### Immunomodulatory Exosomes

In one or more aspects, this disclosure describes compositions including an immunomodulatory exosome and methods of using an immunomodulatory exosome. In one or more embodiments, the immunomodulatory exosome is derived from platelet rich plasma subjected to multiple freeze-thaw cycles and lyophilization. In one or more embodiments, the immunomodulatory exosome may be prepared as described in International Patent Application No. PCT/US2018/065627 (published as International Publication No. WO 2019/118817), U.S. Patent Publication No. 2021/0169812 A1, U.S. Pat. No. 10,596,123, or International Patent Application No. PCT/US2021/054547 (published as International Publication No. WO 2022/081557 A1).

In one or more embodiments, the immunomodulatory exosomes may be a purified exosome product, referred to herein as PEP. PEP is a purified exosome product prepared using a cryodesiccation step that produces a product having a structure that is distinct from exosomes prepared using conventional methods. Production of purified exosome product (PEP) involves separating plasma from blood, isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety. Briefly, PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during conventional exosome preparation methods. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—e.g., a PEP preparation whose character is unchanged by sorting or segregating populations of exosomes in the preparation—naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁺ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation can have both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio in order to avoid uncontrolled cell growth. Conversely, if there is a desire to expand out a clonal population of cells, such as in allogeneic cell-based therapy or immunotherapy, one can increase the ratio of CD63⁺ exosomes in order to ensure that a large population of cells can be derived from a very small source.

Thus, in one or more embodiments, the ratio of CD63⁺ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁺ exosomes to CD63⁻ exosomes may be between 1:1 to 30:1, 2:1 to 20:1, 4:1 to 15:1, or 8:1 to 10:1. In one or more certain embodiments, the PEP product is formulated to contain a 9:1 ratio of CD63⁺ exosomes to CD63⁻ exosomes. In one or more certain embodiments, native PEP, e.g., PEP with an unmodified ratio of CD63⁺ exosomes to CD63⁻ exosomes may be used.

In one or more embodiments, PEP may be modified to include one or more exogenous active agents. As used herein, the term “exogenous” refers to material that is not natively present in the PEP exosomes. Because PEP may be prepared from various starting materials, an active agent may be “exogenous” for PEP prepared from one source material even though it may be endogenous—i.e., natively present—in PEP exosomes prepared from another source. Thus, the evaluation of whether an active agent is exogenous depends on the source material used to prepare PEP. Exemplary exogenous active agents include, but are not limited to, a nucleic acid or a polypeptide. Methods for transforming extracellular vesicles and exemplary exogenous active agents are described in detail in US Patent Application Publication No. US 2021/0259969 A1 and in International Patent Application No. PCT/US2019/043172, which published as International Publication No. WO 2020/023594.

### Monocytes, Macrophages, Microglia, and T cells

Monocytes are circulating myeloid cells that give rise to tissue macrophages and dendritic cells. Regulatory monocytes can regulate immune responses through the production of soluble regulatory factors (including, for example, IL-10, TGF-β, indoleamine 2,3 deoxygenase (IDO), arginase, nitric oxide (NO), etc.), expression of inhibitory or regulatory cell surface molecules (including, for example, PD-L1, PD-L2), by inducing other regulatory cells (including, for example, regulatory T cells (T_{regs})), and/or by enhancing regulatory feed-back loops (VanGundy et al. BMC Immunol. 2014; 15:8).

Macrophages are large mononuclear phagocytic cells differentiated from monocytes. Macrophages can adopt phenotypes ranging from a pro-inflammatory or “M1” phenotype to a less inflammatory or “M2” phenotype that may be associated with the resolution of inflammation (e.g., Spiller et al. Adv. Drug Del. Rev. 2017; 122:74-83). Roles for M1 or M2 macrophages have been suggested in therapeutic strategies ranging from regenerative medicine to cancer immunotherapies.

Microglia are macrophage cells that reside in the central nervous system (CNS). Like peripheral macrophages, microglia can be polarized into “M1” inflammatory and “M2” anti-inflammatory phenotypes (Guo et al., Front Aging Neurosci. 2022 Feb. 16; 14:815347. doi: 10.3389/fnagi.2022.815347. PMID: 35250543; PMCID: PMC8888930).

As used herein, a macrophage or microglial cell exhibiting an “M1-like” phenotype may express or produce one of more of CD14, CD68, CD80, CD86, iNos, CCL2, TNFα, IFNβ, IFNγ, IL-1β, IL-6, IL-12, IL-17, or IL-23. As used herein, a macrophage or microglial cell exhibiting an “M2-like” phenotype expresses or produces one or more of CD14, CD68, CD163, CD206, IL-4, IL-6, IL-10, IL-13, TGF-β, IL-1RA, RETN, or Arginase 1.

Conventional T cells are important lymphocytes in the context of the adaptive immune response. Upon stimulation via their T cell receptor (TCR), in combination with additional co-stimulation signals, T cells become activated and rapidly undergo cell division. Helper T cells express CD3 and CD4 on their surface, and these cells can differentiate into several subsets to defend against various types of pathogens. However, prolonged activation of T cells may lead to chronic inflammation and undesired immunopathology, such as in the case of autoimmunity. Regulatory T cells (Tregs) are a subpopulation of T cells that modulate the immune system, maintain tolerance to self-antigens. In one or more embodiments, Tregs have a CD3⁺CD4⁺CD25^{hi}FoxP3⁺ phenotype. In one or more embodiments, Tregs can further possess a CD127^{lo} phenotype.

### Methods of Making Macrophages and Monocytes

In another aspect, this disclosure describes methods of making an M2-like macrophage, microglial cell, and/or a regulatory monocyte. This disclosure may additionally describe methods of expanding a macrophage, microglial cell, or monocyte.

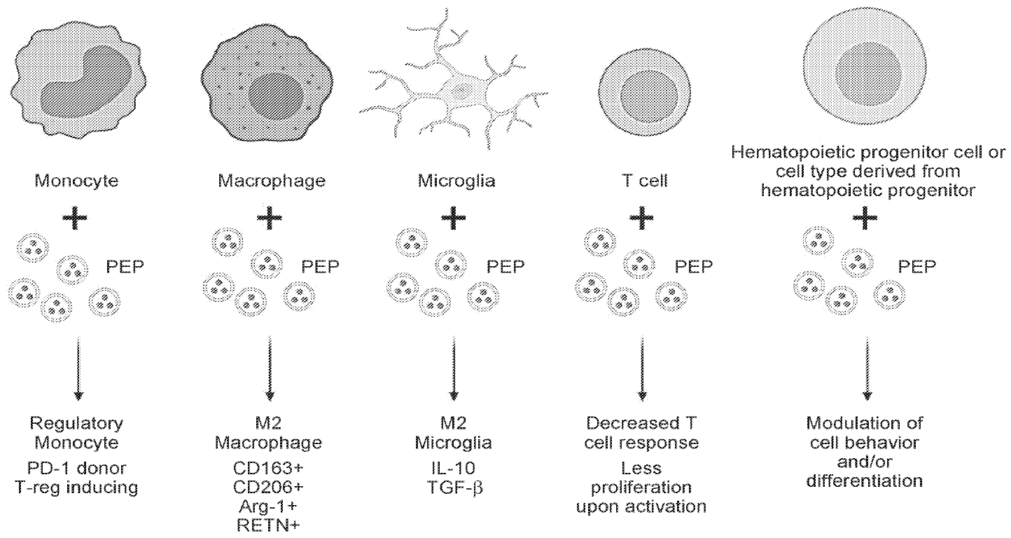

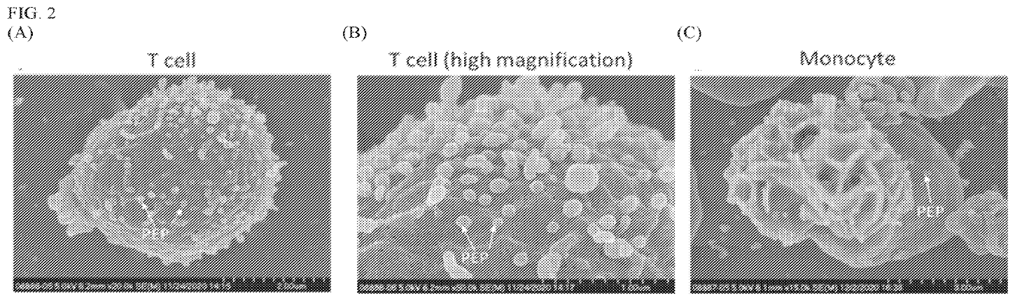

In one or more embodiments, the method includes co-culturing a hematopoietic progenitor cell or a cell differentiated from a hematopoietic cell with an immunomodulatory exosome. FIG. 1. Typically, the hematopoietic progenitor cell or a cell differentiated from a hematopoietic cell can be co-cultured with immunomodulatory exosomes for a period of from 24 hours to 30 days, although methods of making M2-like macrophages, microglial cell, or regulatory monocyte can involve co-culturing the cell with immunomodulatory exosomes for a period of time outside this range.

The hematopoietic progenitor cell or a cell differentiated from a hematopoietic cell can be any cell that can differentiate into a an M2-like macrophage, microglia, or regulatory monocyte. Thus, the hematopoietic progenitor cell can be a myeloid progenitor cell or a lymphocyte progenitor cell. Thus, in one or more embodiments, the method includes deriving a macrophage, microglial cell, or a monocyte or all three from a hematopoietic stem cell source. The hematopoietic stem cell source can include any suitable source of hematopoietic stem cells. Exemplary sources include umbilical cord blood, bone marrow, peripheral blood, etc.

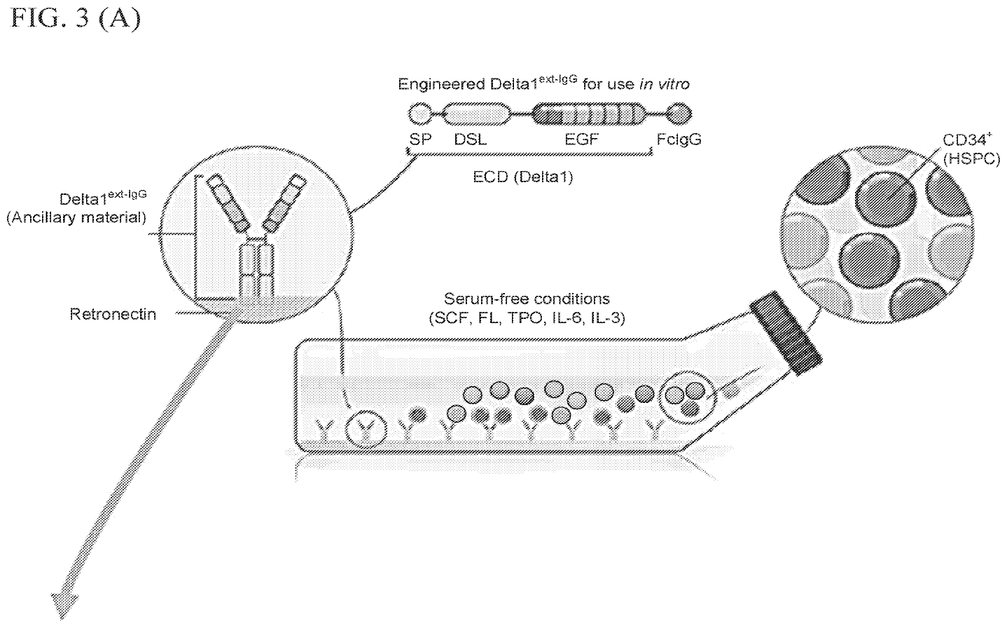

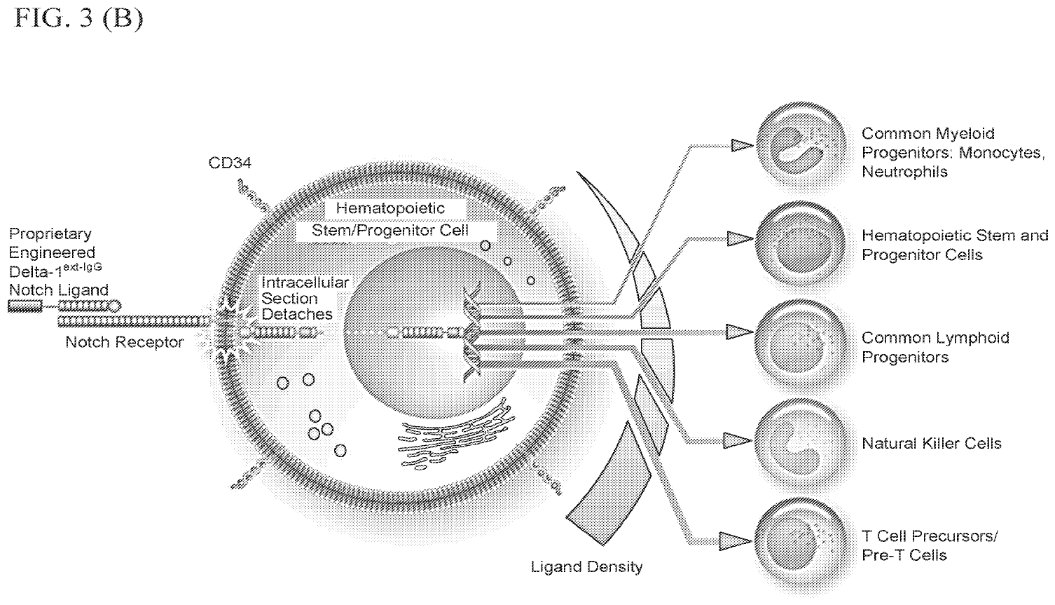

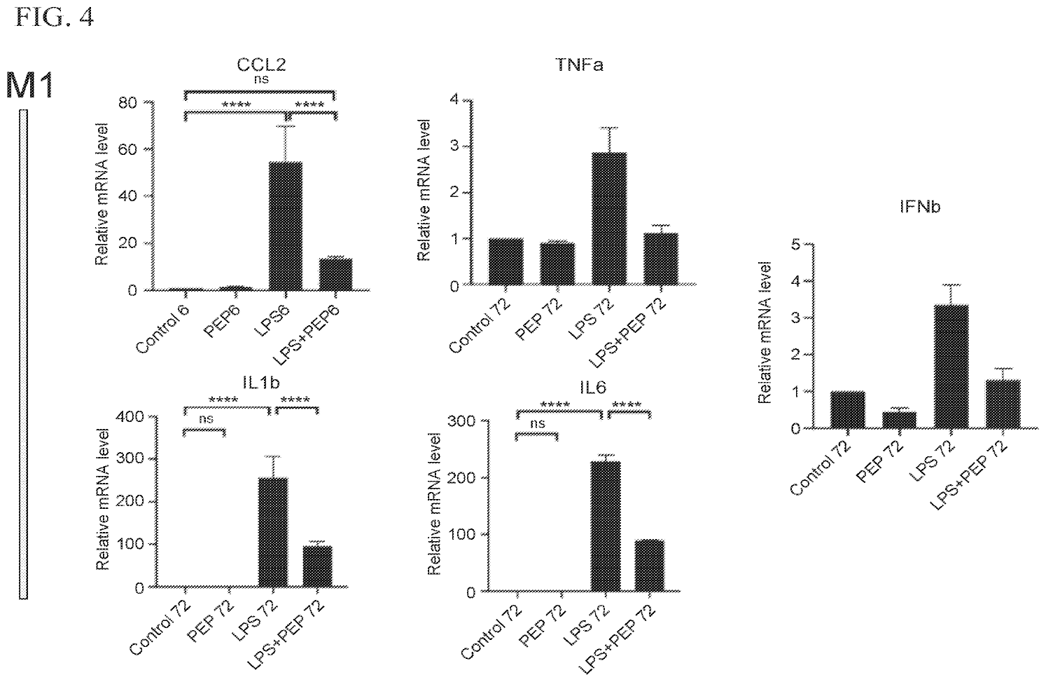

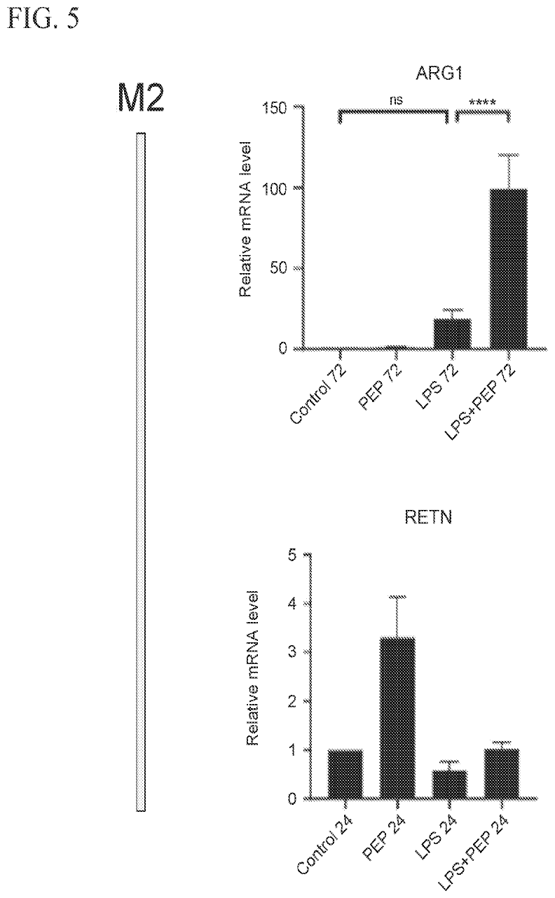

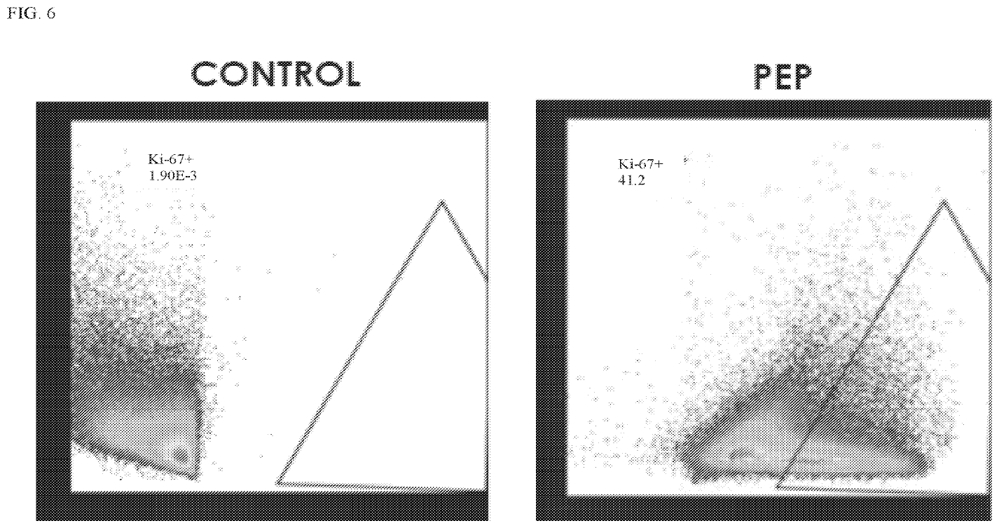

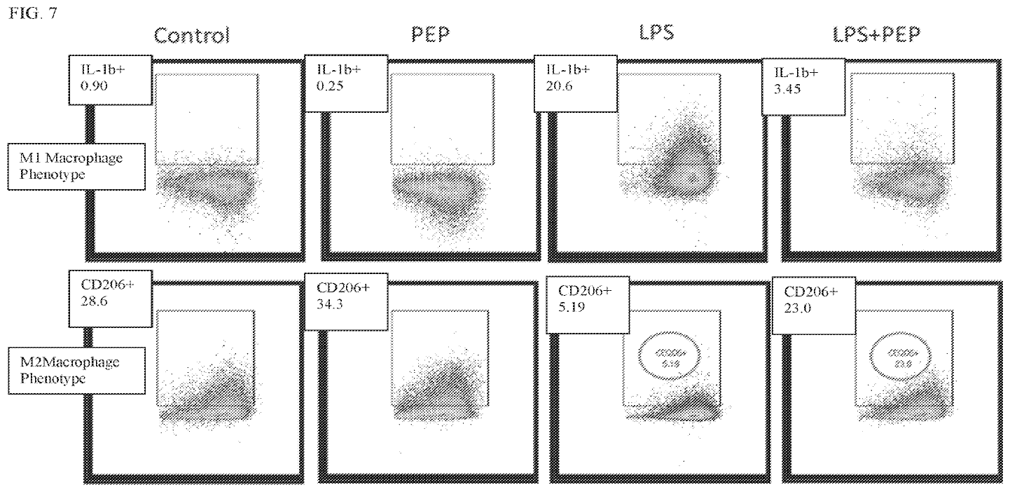

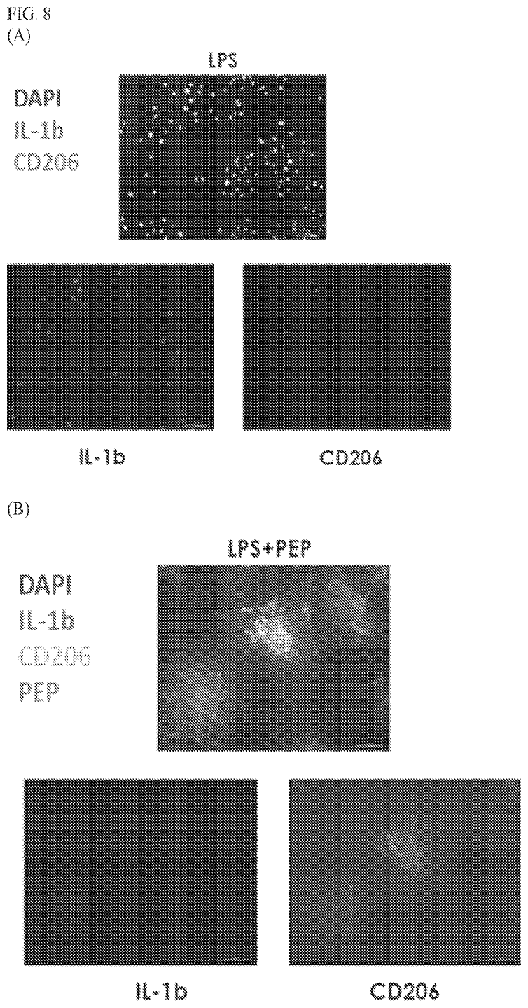

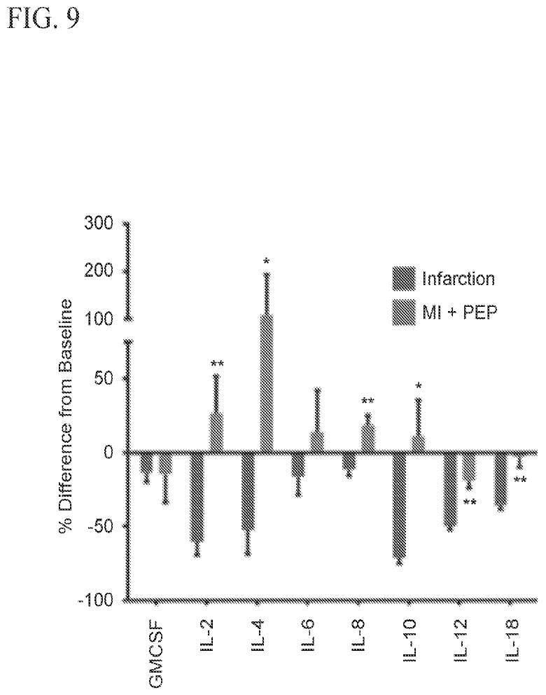

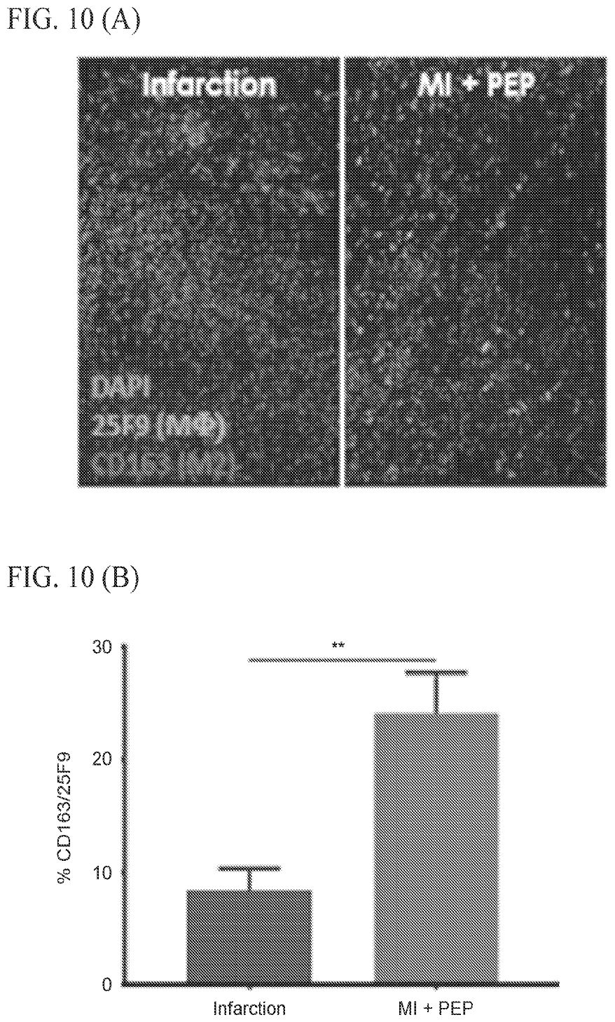

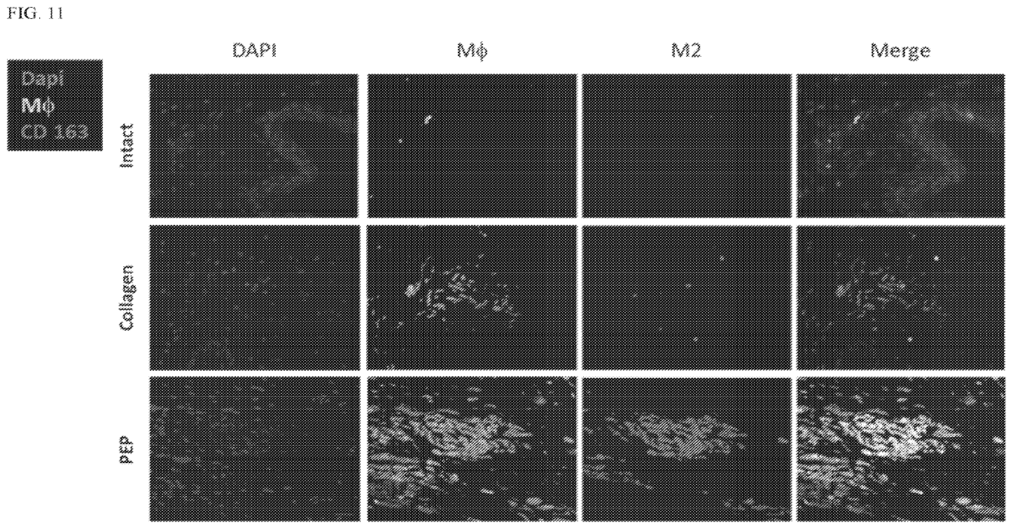

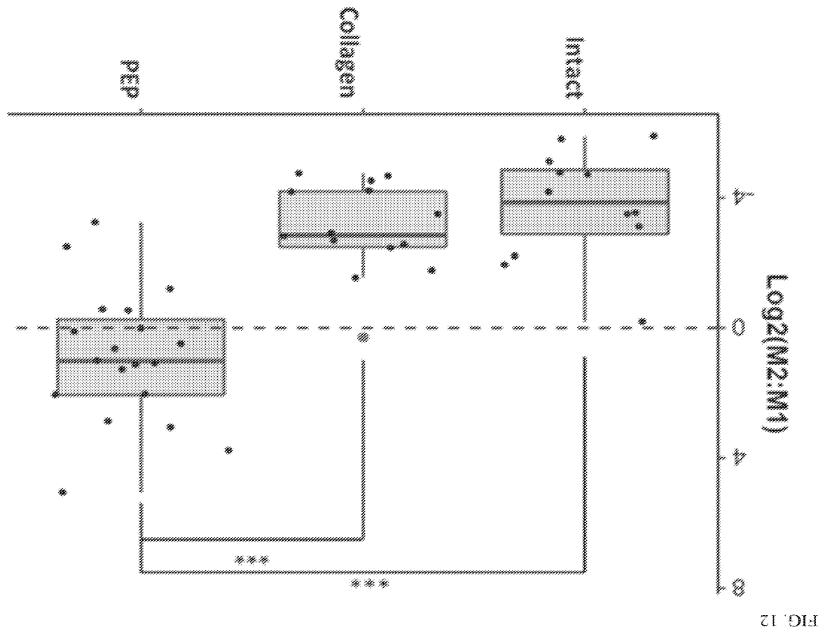

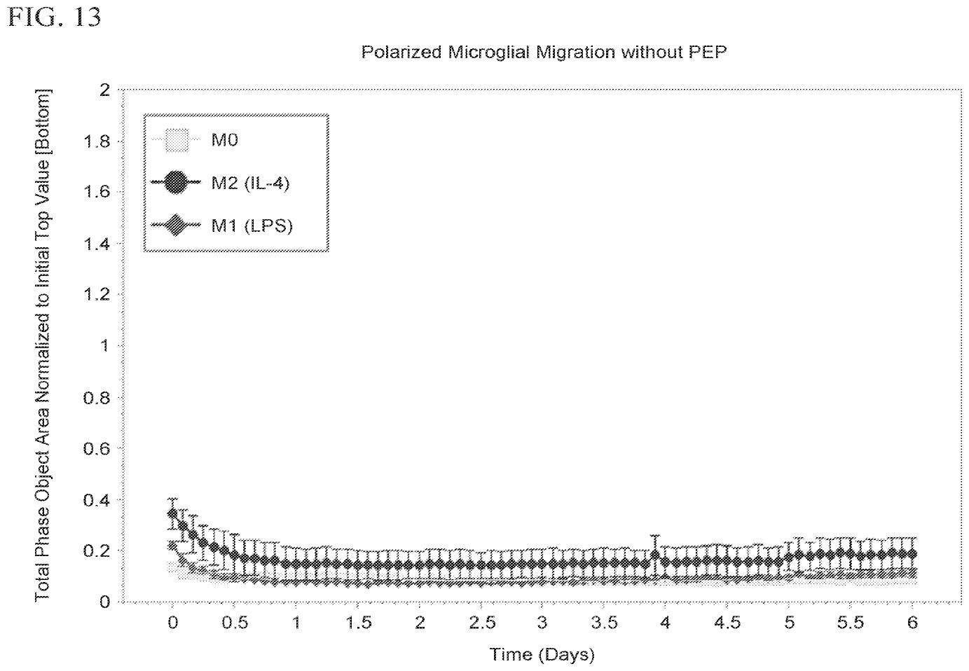

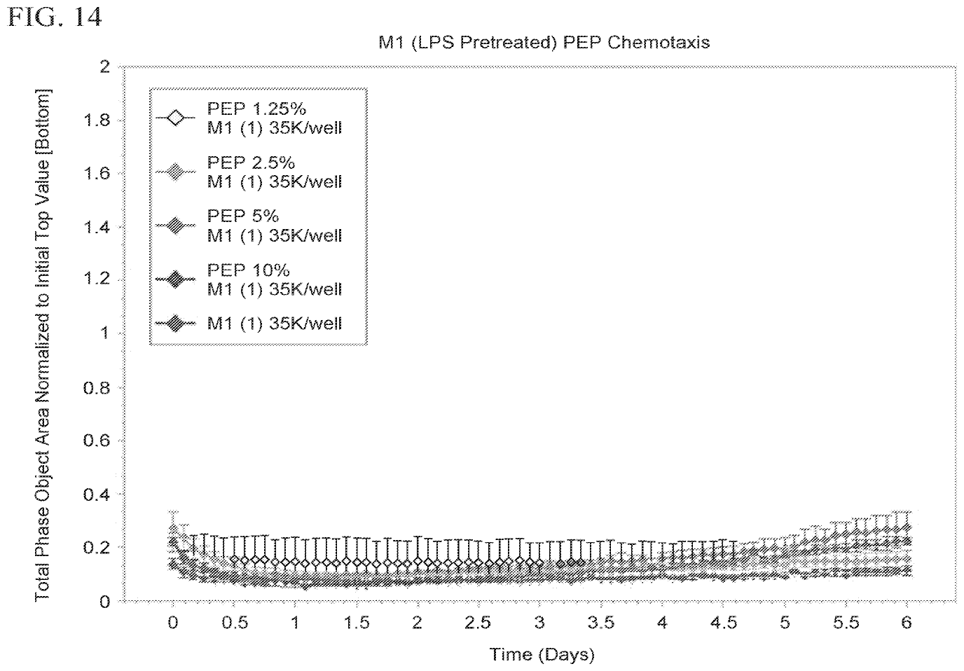

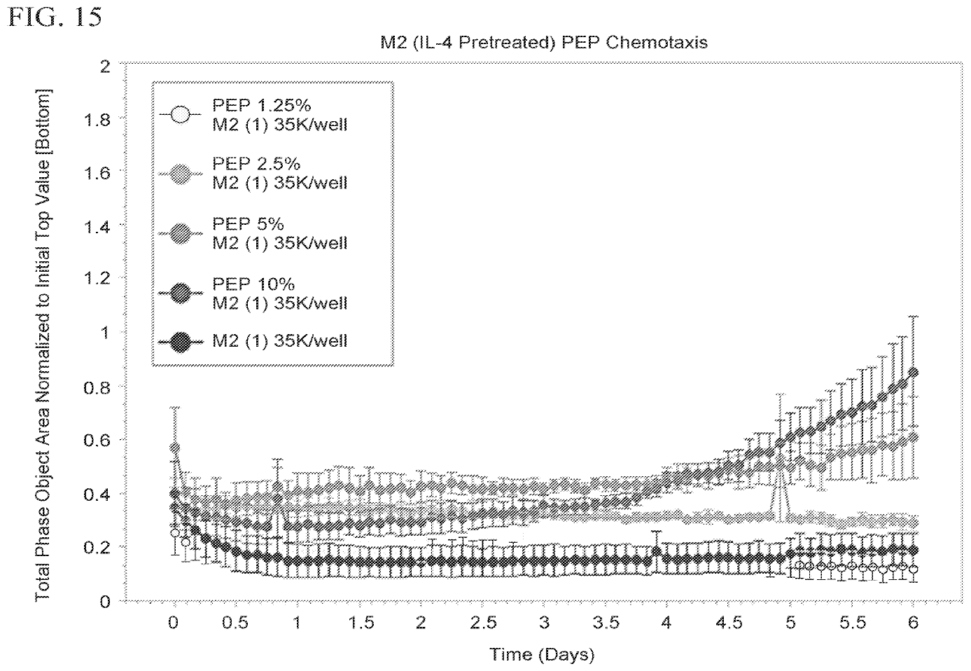

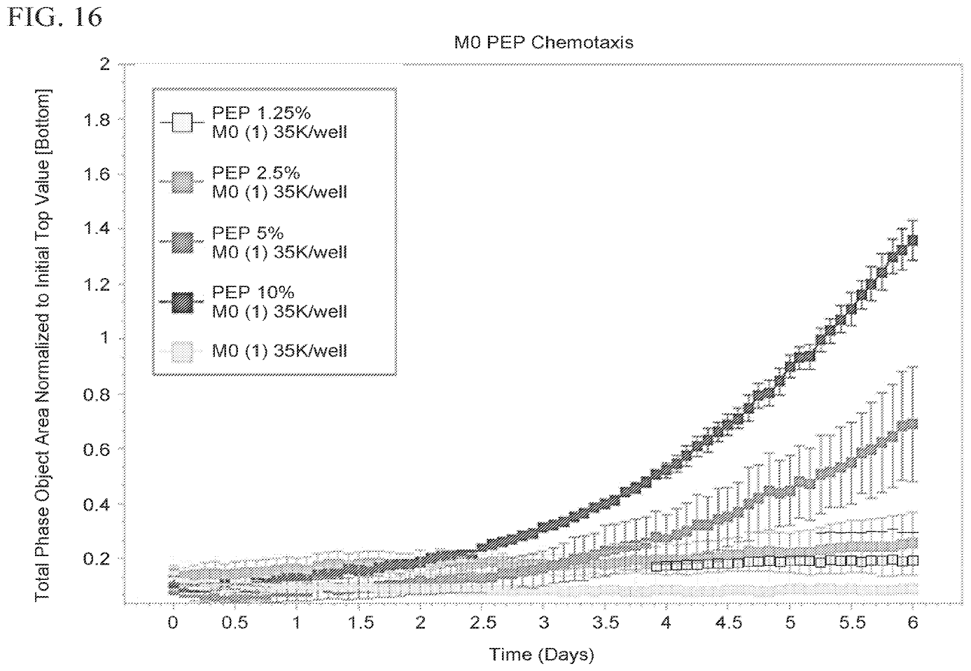

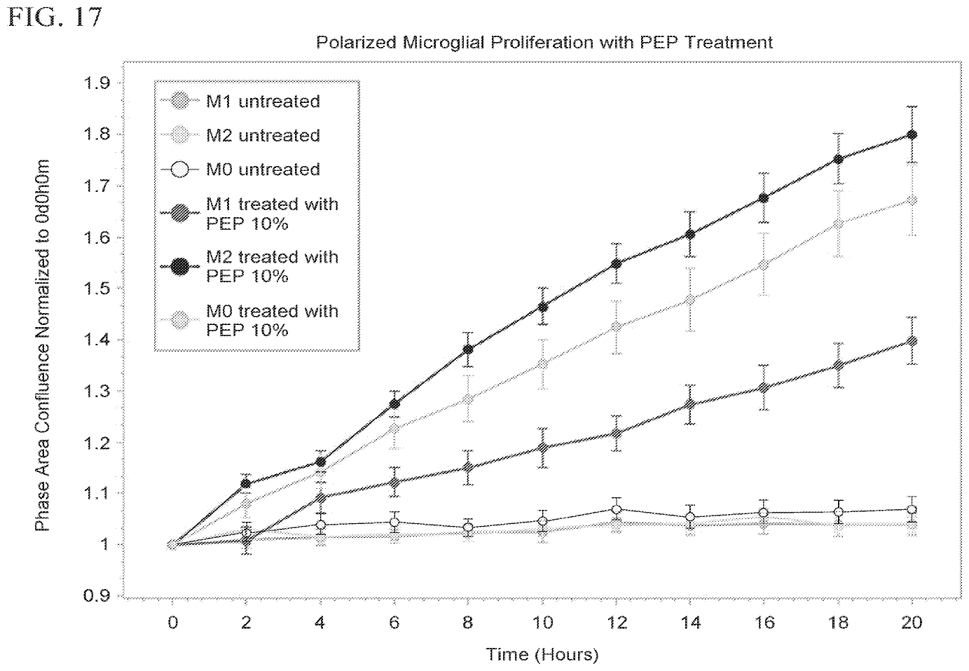

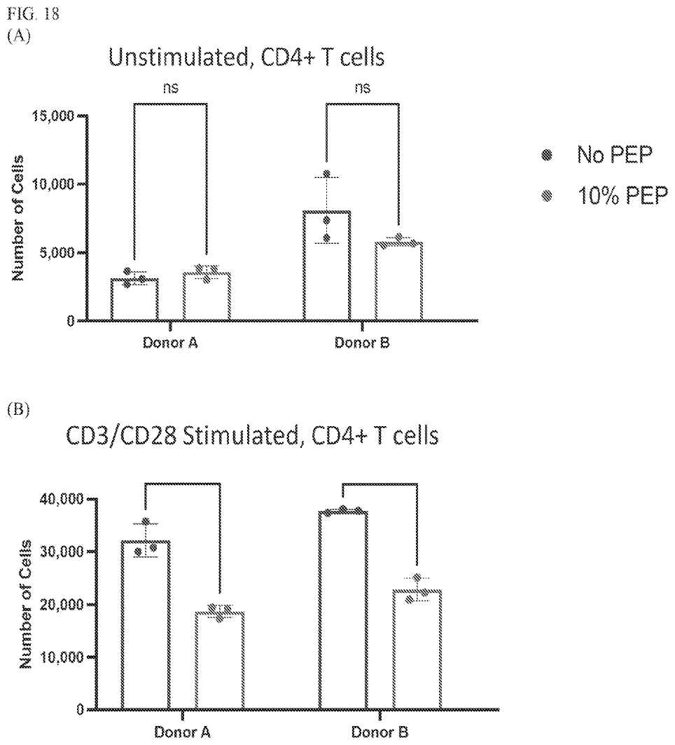

In one or more embodiments, the method deriving a macrophage, microglial cell, a monocyte, or all three from a hematopoietic stem cell source may include culturing a hematopoietic stem cell with a Delta-1 Notch Ligand. FIG. 3A. In one or more embodiments, the Delta-1 Notch Ligand may include dilanubicel (also known as NLA101) from Deverra Therapeutics, Inc. (Seattle, Washington).

In other embodiments, the methods include co-culturing a cell differentiated from a hematopoietic progenitor cell with an immunomodulatory exosome. The cell differentiated from a hematopoietic progenitor cell can be a peripheral blood mononuclear cell (PBMC) or a non-peripheral blood mononuclear cell. Exemplary PBMCs include, but are not limited to, B cells, T cells (including, e.g., CD4⁺ T cells, CD8₊ cells, Tregs, etc.), Natural Killer (NK) cells, macrophages, monocytes, dendritic cells, or myeloid-derived suppressor cells (MDSCs).

In one or more embodiments, PEP may promote expansion of macrophages. In particular, administration of PEP may promote expansion of M2-like macrophages.

### Methods of Using

In another aspect, this disclosure describes methods that involve modulating the immune system using immunomodulatory exosomes. The methods can involve contacting the immunomodulatory exosomes with hematopoietic cells (or cells differentiated from a hematopoietic cell) in vivo or ex vivo. Thus, in one or more embodiments, the methods can include administering a cell differentiated from a hematopoietic cell by contact with immunomodulatory exosomes (i.e., a “treated cell” such as, for example, a monocyte, M2-like macrophage, or other peripheral blood mononuclear cell) to a subject in an amount effective to modulate the subject's immune system. In other embodiments, the methods can include direct administration of immunomodulatory exosomes to a subject in an amount effective to modulate the subject's immune system.

The subject can be a human or a non-human animal such as, for example, a livestock animal, a zoo animal, or a companion animal. Exemplary non-human animal subjects include, but are not limited to, animals that are hominid (including, for example chimpanzees, gorillas, and orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou and reindeer), members of the family Bison (including, for instance, bison), feline (including, for example, tigers, lions, and domesticated cats), canine (including, for example, a wolves and domesticated dogs), avian (including, for example, turkeys, chickens, ducks, and geese), a rodent (including, for example, mice or rats), a member of the family Leporidae (including, for example, rabbits or hares), members of the family Mustelidae (including, for example ferrets), or member of the order Chiroptera (including, for example, bats).

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the infectious agent and regardless of whether the subject may harbor a subclinical amount of the infectious agent. As another example, a subject “at risk” of a non-infectious condition is a subject possessing one or more risk factors associated with the condition such as, for example, genetic predisposition, ancestry, age, sex, geographical location, lifestyle, or medical history. Treatment may also be continued after symptoms have resolved, for example to prevent or delay their recurrence.

Accordingly, treated cells generated as described herein and/or immunomodulatory exosomes can be administered before, during, or after the subject first exhibits a symptom or clinical sign of the condition or, in the case of infectious conditions, before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, in one or more embodiments, the method includes administering an effective amount of treated cells to a subject having, or at risk of having, a particular condition. In other embodiments, the method includes administering an effective amount of immunomodulatory exosomes to a subject having, or at risk of having, a particular condition. In either case, an “effective amount” is an amount effective to reduce, limit progression, ameliorate, or resolve, to any extent, a symptom or clinical sign related to the condition.

In some cases, the subject may be suffering from an inflammation-associated disease state. Exemplary inflammation-associated disease states include, but are not limited to, cardiovascular diseases (e.g., chronic pulmonary respiratory disease, acute respiratory disease, chronic obstructive pulmonary disease (COPD), etc.), neurological diseases, including neuroinflammatory diseases, (e.g., Amyotrophic Lateral Sclerosis (ALS), Alzheimer's Disease, multiple sclerosis, transverse myelitis, neuritis, neurosarcoidosis, Parkinson's Disease, etc.), gastrointestinal diseases (e.g., Crohn's Disease, ulcerative colitis, irritable bowel syndrome, inflammatory bowel disease, etc.), skin diseases (e.g., eczema, dermatitis, psoriasis, etc.), autoimmune diseases (e.g., systemic lupus erythematosus, rheumatoid arthritis, psoriasis, etc.), an inflammatory joint disease, myocarditis, atherosclerosis, diabetes, or osteoporosis. In one or more embodiments, the subject may have a viral infection, such as a SARS-CoV-2 infection. The subject may have a known inflammation-associated disease. Alternately, the subject may be suspected of having an inflammation-associated disease.

In embodiments in which treated cells are administered to a subject, the treated cells generated as described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, hydrogel, scaffold, colloid, and the like. The use of such media and/or agents for pharmaceutical active substances is well known in the art. Except insofar as any conventional media or agent is incompatible with the treated cells, its use in the therapeutic compositions is contemplated. Supplementary active ingredients also can be incorporated into the formulation along with the treated cells. As used herein, “pharmaceutically acceptable” refers to a material that is not biologically or otherwise undesirable, i.e., the material may be administered to an individual along with the treated cells without causing any undesirable biological effects or interacting in a deleterious manner with any of the other components of the pharmaceutical composition in which it is contained.

Treated cells generated as described herein may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, treated cells may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the treated cells into association with a carrier that may include one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of treated cells administered can vary depending on various factors including, but not limited to, the specific treated cells being administered, the weight, physical condition, and/or age of the subject, the site of administration, and/or the route of administration. Thus, the absolute amount of treated cells included in a given unit dosage form can vary widely, and depends upon factors such as the species, age, weight and physical condition of the subject, and/or the method of administration. Accordingly, it is not practical to set forth generally the amount that constitutes an amount of treated cells effective for all possible applications. Those of ordinary skill in the art, however, can readily determine the appropriate amount with due consideration of such factors.

In one or more embodiments, the method can include administering sufficient treated cells to provide a dose of, for example, from about 1×10⁵ to about 1×10¹⁰ cells to the subject, although in one or more embodiments the methods may be performed by administering treated cells in a dose outside this range. A dose can be determined either as a total number of cells or as a ratio of cells per body weight (cells/kg). Absolute minimum dose should be 1×10³ cells. Absolute maximum dose should be approximately 1×10¹⁵. Typically, a dose of 1×10⁵ to about 1×10¹⁰ cells can be administered up to three times a day.

A single dose may be administered all at once, continuously for a prescribed period of time, or in multiple discrete administrations. When multiple administrations are used, the amount of each administration may be the same or different. For example, a dose of 2×10¹⁰ per day may be administered as a single administration of 2×10¹⁰, may be administered continuously over 24 hours, may be administered as two 1×10¹⁰ administrations, or as a first administration of 1.5×10¹⁰ followed by a second administration of 0.5×10¹⁰. When multiple administrations are used to deliver a single dose, the interval between administrations may be the same or different.

In one or more embodiments, the treated cells may be administered, for example, from a single dose to multiple doses per week, although in one or more embodiments the method can involve a course of treatment that includes administering doses of the treated cells at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In certain embodiments, treated cells may be administered to the subject from about once per month to about five times per week.

In one or more embodiments, the treated cell is an M2-like macrophage. In some cases, the M2-like macrophage expresses CD163 and/or CD206. In some cases, the M2-like macrophage further expresses IL-4, IL-10, TGF-β, IL-IRA, RETN, or Arginase 1, or a combination of two or more thereof.

In embodiments in which immunomodulatory exosomes are administered to a subject, the immunomodulatory exosomes may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, hydrogel, scaffold, colloid, and the like. The use of such media and/or agents for pharmaceutical active substances is well known in the art. Except insofar as any conventional media or agent is incompatible with the immunomodulatory exosomes, its use in the therapeutic compositions is contemplated. Supplementary active ingredients also can be incorporated into the formulation along with the immunomodulatory exosomes. As used herein, “pharmaceutically acceptable” refers to a material that is not biologically or otherwise undesirable, i.e., the material may be administered to an individual along with the immunomodulatory exosomes without causing any undesirable biological effects or interacting in a deleterious manner with any of the other components of the pharmaceutical composition in which it is contained.

Immunomodulatory exosomes may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, immunomodulatory exosomes may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the immunomodulatory exosomes into association with a carrier that may include one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of immunomodulatory exosomes administered can vary depending on various factors including, but not limited to, the specific immunomodulatory exosomes being administered, the weight, physical condition, and/or age of the subject, the site of administration, and/or the route of administration. Thus, the absolute amount of immunomodulatory exosomes included in a given unit dosage form can vary widely, and depends upon factors such as the species, age, weight and physical condition of the subject, and/or the method of administration. Accordingly, it is not practical to set forth generally the amount that constitutes an amount of immunomodulatory exosomes effective for all possible applications. Those of ordinary skill in the art, however, can readily determine the appropriate amount with due consideration of such factors.

In one or more embodiments, the method can include administering sufficient immunomodulatory exosomes to provide a dose of, for example, from about 1×10⁵ to about 1×10¹⁶ to the subject, although in one or more embodiments the methods may be performed by administering immunomodulatory exosomes in a dose outside this range. A dose can be determined either as a total number of immunomodulatory exosomes or as a ratio of immunomodulatory exosomes per body weight (exosomes/kg). In one or more embodiments, PEP may be dosed on a per weight bases, either as total mg dose or mg per body weight (mg/kg). In one or more embodiments, the minimum dose delivered is 1×10³ immunomodulatory exosomes. In one or more embodiments, the maximum dose delivered is 1×10²⁰ immunomodulatory exosomes Typically, a dose of 1×10⁵ immunomodulatory exosomes to about 1×10¹⁶ immunomodulatory exosomes can be administered up to three times a day.

A single dose may be administered all at once, continuously for a prescribed period of time, or in multiple discrete administrations. When multiple administrations are used, the amount of each administration may be the same or different. For example, a dose of 2×10¹⁰ per day may be administered as a single administration of 2×10¹⁰, may be administered continuously over 24 hours, may be administered as two administrations of 1×10¹⁰ immunomodulatory exosomes, or as a first administration of 1.5×10¹⁰ immunomodulatory exosomes followed by a second administration of 0.5×10¹⁰ immunomodulatory exosomes. When multiple administrations are used to deliver a single dose, the interval between administrations may be the same or different.

In one or more embodiments, the immunomodulatory exosomes may be administered, for example, from a single dose to multiple doses per week, although in one or more embodiments the method can involve a course of treatment that includes administering doses of the immunomodulatory exosomes at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In certain embodiments, immunomodulatory exosomes may be administered to the subject from about once per month to about five times per week.

In one or more embodiments, the method further includes administering one or more additional therapeutic agents. The one or more additional therapeutic agents may be administered before, after, and/or coincident to the administration of immunomodulatory exosomes or treated cells. The additional therapeutic agent or agents may be co-administered with the immunomodulatory exosomes or treated cells. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that would not exist in a chemical or physical mixture with one another. In other embodiments, the immunomodulatory exosomes or treated cells and the additional therapeutic agent may be administered as part of a mixture or cocktail. In some aspects, the administration of the immunomodulatory exosomes or treated cells may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include, but are not limited to, a monoclonal antibody, an antibody fragment, a multi-specific antibody-drug conjugate, a non-steroidal anti-inflammatory drug (NSAID), a corticosteroid, an antibiotic, an anti-viral (e.g., remdesivir or oseltamivir), or convalescent plasma. Exemplary therapeutic monoclonal antibodies include, but are not limited to, remdesivir, baricitinib, adalimumab, sotrovimab, benralizumab, raxibacumab, or guselkumab.

In several places throughout the above description, guidance is provided through lists of examples, which examples can be used in various combinations. In each instance, the recited list serves only as a representative group and should not be interpreted as an exclusive list.

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

In the preceding description, the words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

### EXEMPLARY EMBODIMENTS

Embodiment 1 is a method comprising:
culturing a hematopoietic progenitor cell or cell differentiated from a hematopoietic progenitor cell with an immunomodulatory exosome to produce a treated cell,
wherein the immunomodulatory exosome is derived from platelet rich plasma subjected to multiple freeze-thaw cycles and lyophilization.

Embodiment 2 is the method of Embodiment 1, wherein the hematopoietic progenitor cell comprises a myeloid progenitor cell.

Embodiment 3 is the method of Embodiment 1, wherein the hematopoietic progenitor cell comprises a lymphocyte progenitor cell.

Embodiment 4 is the method of Embodiment 1, wherein the cell differentiated from a hematopoietic progenitor cell is a peripheral blood mononuclear cell (PBMC).

Embodiment 5 is the method of Embodiment 4, wherein the PBMC comprises a myeloid cell.

Embodiment 6 is the method of Embodiment 5, wherein the myeloid cell comprises a monocyte or a macrophage.

Embodiment 7 is the method of Embodiment 4, wherein the PBMC comprises a lymphocyte.

Embodiment 8 is the method of Embodiment 7, wherein the lymphocyte comprises a B cell, a T cell, or an NK cell.

Embodiment 9 is the method of Embodiment 1, wherein the treated cell comprises a monocyte or a macrophage.

Embodiment 10 is the method of any preceding Embodiment, further comprising administering the treated cell to a subject.

Embodiment 11 is the method of Embodiment 10, wherein the subject is suffering from an inflammation-associated disease state.

Embodiment 12 is the method of Embodiment 11, wherein the inflammation-associated disease state comprises a chronic pulmonary respiratory disease, an acute respiratory disease, an inflammatory joint disease, atherosclerosis, diabetes, cardiovascular disease, inflammatory bowel disease, neuroinflammatory disease, or osteoporosis.

Embodiment 13 is the method of Embodiment 11, wherein the inflammation-associated disease state comprises infection with SARS-CoV-2.

Embodiment 14 is the method of any preceding Embodiment, wherein the treated cell comprises an M2 macrophage.

Embodiment 15 is the method of Embodiment 14, wherein the M2 macrophage expresses CD163.

Embodiment 16 is the method of any preceding Embodiment, wherein the treated cell expresses CD14 or CD206 or both.

Embodiment 17 is the method of any preceding Embodiment, wherein the treated cell expresses IL-10, TGF-β, IL-1ra, or Arginase 1, or a combination thereof.

Embodiment 18 is a composition comprising immunomodulatory exosomes for use in treating an inflammation-associated disease state in a subject,
wherein the immunomodulatory exosomes are derived from platelet rich plasma subjected to multiple freeze-thaw cycles and lyophilization.

Embodiment 19 is the composition of Embodiment 18, wherein the inflammation-associated disease state comprises a chronic pulmonary respiratory disease, an acute respiratory disease, an inflammatory joint disease, atherosclerosis, diabetes, cardiovascular disease, inflammatory bowel disease, neuroinflammatory disease, or osteoporosis.

Embodiment 20 is the composition of Embodiment 19, wherein the inflammation-associated disease state comprises infection with SARS-CoV-2.

Embodiment 21 is the composition of Embodiment 19, wherein the inflammation-associated disease state comprises chronic obstructive pulmonary disease (COPD).

Embodiment 22 is the composition of Embodiment 19, wherein the inflammation-associated disease state comprises myocarditis.

Embodiment 23 is the composition of any one of Embodiments 18-22, wherein the composition is formulated for intravenous administration.

Embodiment 24 is the composition of any one of Embodiments 18-22, wherein the composition is formulated for intramuscular administration.

Embodiment 25 is the composition of any one of Embodiments 18-22, wherein the composition is formulated for intraperitoneal administration.

Embodiment 26 is the composition of any of Embodiments 18-25, wherein the composition further comprises one or more additional active agents.

Embodiment 27 is the composition of any of Embodiments 18-26, wherein the composition is for use for treating a domestic animal, a domesticated animal, a zoo animal, or a human.

Embodiment 28 is a method of treating an inflammation-associated disease state in a subject, the method comprising:
administering an immunomodulatory exosome derived from platelet rich plasma subjected to multiple freeze-thaw cycles and lyophilization to the subject.

Embodiment 29 is the method of Embodiment 28, wherein the inflammation-associated disease state comprises a chronic pulmonary respiratory disease, an acute respiratory disease, an inflammatory joint disease, atherosclerosis, diabetes, cardiovascular disease, inflammatory bowel disease, neuroinflammatory disease, or osteoporosis.

Embodiment 30 is the method of Embodiment 29, wherein the inflammation-associated disease state comprises infection with SARS-CoV-2.

Embodiment 31 is the method of Embodiment 29, wherein the inflammation-associated disease state comprises chronic obstructive pulmonary disease (COPD).

Embodiment 32 is the method of Embodiment 29, wherein the inflammation-associated disease state comprises myocarditis.

Embodiment 33 is the method of any of Embodiments 28-32, wherein the method comprises administering a composition comprising the immunomodulatory exosome, wherein the composition is formulated for intravenous administration.

Embodiment 34 is the method of any of Embodiments 28-32, wherein the method comprises administering a composition comprising the immunomodulatory exosome, wherein the composition is formulated for intramuscular administration.

Embodiment 35 is the method of any of Embodiments 28-32, wherein the method comprises administering a composition comprising the immunomodulatory exosome, wherein the composition is formulated for intraperitoneal administration.

Embodiment 36 is the method of any of Embodiments 28-35, wherein administering the immunomodulatory exosome promotes expansion of Treg cells.

Embodiment 37 is the method of Embodiment 36, wherein the Treg cells have a phenotype comprising CD3⁺CD4⁺CD25^{hi}FoxP3⁺.

Embodiment 38 is the method of Embodiment 37, wherein the Treg has a phenotype that further includes CD127^{lo}.

Embodiment 39 is the method of any of Embodiments 28-38, wherein administering the immunomodulatory exosome promotes expansion of M2 macrophages.

Embodiment 40 is the method of Embodiment 39, wherein the M2 macrophage expresses CD163.

Embodiment 41 is the method of Embodiment 39 or Embodiment 40, wherein the M2 macrophage does not express CD80.

Embodiment 42 is the method of any of Embodiments 39-41, wherein the M2 macrophage expresses CD14 or CD206 or both.

Embodiment 43 is the method of any of Embodiments 28-42, wherein the method further comprises co-administering an additional therapeutic agent.

Embodiment 44 is the method of Embodiment 43, wherein the additional therapeutic agent comprises an immunomodulatory monoclonal antibody or immunomodulatory small-molecule.

### EXAMPLES

The present invention is illustrated by the following examples. The particular examples, materials, amounts, and procedures are to be interpreted broadly in accordance with the scope and spirit of the invention as set forth herein.

### Scanning Electron Microscopy (SEM)

Human peripheral blood mononuclear cells (PBMCs) were isolated from whole blood and resuspended in human plasma. One vial of PEP (75 mg) was reconstituted in 1 mL of sterile saline. The PEP and PBMCs were then mixed and gently agitated for 30 to 60 minutes. After co-incubation, samples were washed using centrifugation and sterile PBS. The supernatant was removed, and the sample was resuspended in Trump's fixative for electron microscopy.

### qPCR

Cells were using Trizol reagent and RNA was isolated using an RNA isolation spin column. After quantification and quality assessment using a spectrophotometer (NANODROP 2000, Thermo Fisher Scientific, Inc., Waltham, MA), RNA samples were reverse transcribed into cDNA. Gene expression was then quantified by real-time quantitative PCR with a SYBR GREEN RT-qPCR Kit. Reactions were measured with a quantitative real-time thermal cycler. Transcript levels were calculated using the 2^{ΔΔCt} method and normalized to a housekeeping gene.

### Immunohistochemistry (IHC)

Formalin-fixed samples were embedded in paraffin and sectioned on a microtome into 10 μm sections. Sections were then de-paraffinized in sequential xylene washes and rehydrated in decreasing amounts of ethanol baths, finally being washed in water. Antigen retrieval was conducted by submerging sections into a sodium citrate buffer (10 mM sodium citrate, 0.05% Tween 20, pH 6.0) and boiling for 10 minutes in a pressure cooker. Sections were then permeabilized with blocking buffer (PBS+5% normal donkey serum, 5% BSA, 0.2% Triton-X) for an hour at room temperature. Primary antibodies against MΦ, and M2 macrophages were diluted in blocking buffer overnight at 4° C. Then, secondary antibodies (ALEXA FLUOR, Thermo Fisher Scientific, Inc., Waltham, MA) were diluted 1:500 in blocking buffer and incubated for one hour at room temperature. Following washes, PROLONG Gold Antifade Mountant with DAPI (Thermo Fisher Scientific, Inc., Waltham, MA) was added to sections, a cover glass applied, and imaged on an inverted fluorescent microscope (AXIO OBSERVER, Carl ZEISS AG, Oberkochen, Germany) with variable fluorescence objectives.

### Murine Monocyte In Vitro Culture

Peripheral blood mononuclear cells (PBMCs) were isolated from mouse blood. CD14⁺ cells were treated with PEP and/or LPS. Treated cells were harvested for analysis.

### Murine Macrophage In Vitro Culture

Mice were sacrificed in accordance with IACUC standards. Femurs were harvested from sacrificed mice and flushed with PBS to isolate bone marrow cells. Bone marrow cells were washed and cultured in media containing macrophage colony-stimulating factor to promote macrophage differentiation. Adherent macrophages were harvested and treated with PEP and/or LPS, then harvested for analysis.

### Flow Cytometry of Murine Monocytes or Macrophages

Treated murine monocytes or macrophages were harvested and washed in PBS. Cells were stained with fluorescent conjugated antibodies for detection via flow cytometry (anti-Ki-67 for proliferation, anti-IL-1β for M1 macrophage identification, and anti-CD206 for M2 macrophage identification). After incubation with fluorescent antibodies, cells were washed and analyzed on a flow cytometer.

### Microglia Chemotaxis

HMC3 cells (ATCC: CRL-3304; American Type Culture Collection, Manassas, VA) were plated on a 6-well tissue culture plate at a density of (3×10⁵ cells/well) with EMEM supplemented with 10% FBS for 24 hours and incubated at 37° C. and 5% CO₂. After 24 hours, two wells were treated with 20 ng/mL of recombinant human IL-4 (R&D Systems, Inc., Minneapolis, MN). 24 hours later, two more wells were treated with 10 ng/mL LPS (Sigma-Aldrich, St. Louis, MO). After another 24 hours, all wells were washed with PBS and treated with serum-free minimal essential medium (EMEM). The cells were then trypsinized and seeded onto a 96-well plate (INCUCYTE Clearview, Essen Bioscience, Inc., Ann Arbor, MI) for chemotaxis at a density of 3×10³ cells/well. The bottom reservoir was filled with PBS and placed back in the incubator to allow cell adhesion. Two hours later, the bottom reservoir for each group was replaced with serum-free EMEM or a range of PEP dilutions (1.5%, 2.5%, 5%, and 10%) in serum-free EMEM. The plate was monitored for six days in an incubator (Sartorius AG, Göttingen, Germany) and analyzed using live-cell analysis system (Sartorius AG, Göttingen, Germany).

### Microglia Proliferation

HMC3 cells (ATTC: CRL-3304; American Type Culture Collection, Manassas, VA) were plated on a six-well tissue culture plate at a density of (3×10⁵ cells/well) with minimal essential medium (EMEM) supplemented with 10% FBS for 24 hours and incubated at 37° C. and 5% CO₂. After 24 hours, two wells were treated with 20 ng/ml of human recombinant IL-4 (R&D Systems, Inc., Minneapolis, MN). 24 hours later, two more wells were treated with 10 ng/mL LPS (Sigma-Aldrich, St. Louis, MO). After another 24 hours, the cells were washed with PBS, and then half of the wells were treated with serum-free EMEM, and the remaining wells were treated with 10% PEP in serum-free media. The plate was monitored for six days in an incubator (Sartorius AG, Göttingen, Germany) and analyzed using live-cell analysis system (Sartorius AG, Göttingen, Germany).

### Flow Cytometry on Human T Cells

Peripheral blood mononuclear cells (PBMCs) were isolated from apheresis blood cones via FICOLL (Cytiva, Marlborough, MA) gradient. Total PBMCs were plated in the absence (no stimulation) or presence (stimulation) of CD3/CD28 stimulator and the absence or presence of PEP for a total of seven days. After seven days, cells were harvested and stained with fluorescent conjugated antibodies for detection via flow cytometry. An anti-CD45 antibody was used to detect leukocytes, an anti-CD3 antibody was used to detect T cells, and an anti-CD4 antibody was used to identify CD4+ helper T cells. Cells were washed and analyzed on a flow cytometer.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “approximately” or “about.” Accordingly, unless otherwise indicated to the contrary, the numerical parameters set forth in the specification and claims are approximations that may vary depending upon the desired properties sought to be obtained by the present invention. Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. At the very least, and not as an attempt to limit the doctrine of equivalents to the scope of the claims, each numerical parameter should at least be construed in light of the number of reported significant digits and by applying ordinary rounding techniques.

All headings are for the convenience of the reader and should not be used to limit the meaning of the text that follows the heading, unless so specified.

1. A method comprising:
culturing a hematopoietic progenitor cell or cell differentiated from a hematopoietic progenitor cell with an immunomodulatory exosome to produce a treated cell,
wherein the immunomodulatory exosome is derived from platelet rich plasma subjected to multiple freeze-thaw cycles and lyophilization.

2. The method of claim 1, wherein the hematopoietic progenitor cell comprises a myeloid progenitor cell.

3. The method of claim 1, wherein the hematopoietic progenitor cell comprises a lymphocyte progenitor cell.

4. The method of claim 1, wherein the cell differentiated from a hematopoietic progenitor cell is a peripheral blood mononuclear cell (PBMC).

5. The method of claim 4, wherein the PBMC comprises a myeloid cell.

6. The method of claim 5, wherein the myeloid cell comprises a monocyte or a macrophage.

7. The method of claim 4, wherein the PBMC comprises a lymphocyte.

8. The method of claim 7, wherein the lymphocyte comprises a B cell, a T cell, or an NK cell.

9. The method of claim 1, wherein the treated cell comprises a monocyte or a macrophage.

10. The method of claim 1, further comprising administering the treated cell to a subject.

11. The method of claim 10, wherein the subject is suffering from an inflammation-associated disease state.

12. The method of claim 11, wherein the inflammation-associated disease state comprises a chronic pulmonary respiratory disease, an acute respiratory disease, an inflammatory joint disease, atherosclerosis, diabetes, cardiovascular disease, inflammatory bowel disease, neuroinflammatory disease, or osteoporosis.

13. The method of claim 11, wherein the inflammation-associated disease state comprises infection with SARS-CoV-2.

14. The method of claim 1, wherein the treated cell comprises an M2 macrophage.

15. The method of claim 14, wherein the M2 macrophage expresses CD163.

16. The method of claim 1, wherein the treated cell expresses CD14 or CD206 or both.

17. The method of claim 1, wherein the treated cell expresses IL-10, TGF-Bβ, IL-1ra, or Arginase 1, or a combination thereof.

18-27. (canceled)

28. A method of treating an inflammation-associated disease state in a subject, the method comprising:
administering an immunomodulatory exosome derived from platelet rich plasma subjected to multiple freeze-thaw cycles and lyophilization to the subject.

29. The method of claim 28, wherein the inflammation-associated disease state comprises a chronic pulmonary respiratory disease, an acute respiratory disease, an inflammatory joint disease, atherosclerosis, diabetes, cardiovascular disease, inflammatory bowel disease, neuroinflammatory disease, or osteoporosis.

30. The method of claim 29, wherein the inflammation-associated disease state comprises infection with SARS-CoV-2.

31. The method of claim 29, wherein the inflammation-associated disease state comprises chronic obstructive pulmonary disease (COPD).

32. The method of claim 29, wherein the inflammation-associated disease state comprises myocarditis.

33. The method of claim 28, wherein the method comprises administering a composition comprising the immunomodulatory exosome, wherein the composition is formulated for intravenous administration.

34. The method of claim 28, wherein the method comprises administering a composition comprising the immunomodulatory exosome, wherein the composition is formulated for intramuscular administration.

35. The method of claim 28, wherein the method comprises administering a composition comprising the immunomodulatory exosome, wherein the composition is formulated for intraperitoneal administration.

36. The method of claim 28, wherein administering the immunomodulatory exosome promotes expansion of Treg cells.

37. The method of claim 36, wherein the Treg cells have a phenotype comprising CD3⁺CD4⁺CD25^{hi}FoxP3⁺.

38. The method of claim 37, wherein the Treg has a phenotype that further includes CD127^{lo}.

39. The method of claim 28, wherein administering the immunomodulatory exosome promotes expansion of M2 macrophages.

40. The method of claim 39, wherein the M2 macrophage expresses CD163.

41. The method of claim 39, wherein the M2 macrophage does not express CD80.

42. The method of claim 39, wherein the M2 macrophage expresses CD14 or CD206 or both.

43. The method of claim 28, wherein the method further comprises co-administering an additional therapeutic agent.

44. The method of claim 43, wherein the additional therapeutic agent comprises an immunomodulatory monoclonal antibody or immunomodulatory small molecule.
